(19) United States
(12) Patent Application Publication
(10) Pub. No.: US 2008/0107735 A1
(43) Pub. Date: May 8, 2008

(54) Macrocyclic formulations for transmembrane drug delivery

(76) Inventors: Robert J. Gyurik, Exeter, NH (US); Carl Reppucci, North Andover, MA (US)

Correspondence Address: Raymond E. Stauffer, Esq.;c/o Carella, Byrne, Bain, Gilfillan,, Cecchi, Stewart & Olstein, 5 Becker Farm Road, Roseland, NJ, 07068, US

(21) Appl. No.: 11/591,788

(22) Filed: Nov. 2, 2006

(51) Int. Cl. A61K 9/14

(52) U.S. Cl. 424/486

(57) ABSTRACT

Topically applied pharmaceutical products comprising (a) an active ingredient, (b) an organogel, and (c) a non-irritating permeation enhancer, are disclosed, along with and methods of use and formulations thereof. The topically applied products and methods are intended to be used to enhance any of the transdermal, subdermal, transmembrane, musculoskeletal, transungual, transonychial, and/or peronychial deliveries of a therapeutically effective amount of an active ingredient to a patient. The invention is particularly directed to topically applied pharmaceutical products for enhancing the delivery of a therapeutically effective amount of an active ingredient to a diseased or infected nail bed, nail matrix, and/or nail plate in the toenails or fingernails of a patient suffering from onychomycosis. The invention is also directed to delivery of an anti-inflammatory agent, preferably of the Cox-2 enzyme inhibitory class, topically to a site of soft tissue injury or discomfort.

This application claims the priority of U.S. Provisional Patent Application Ser. No. ______,__ filed on Nov. 2, 2005, which Provisional Patent Application is converted U.S. Utility Patent application Ser. No. 11/265,711, filed on Nov. 2, 2005, which Utility Patent Application was converted to such Provisional Patent Application by Request Under 37 CFR 1.53(c)(2), filed Oct. 30, 2006, the disclosures of which application(s) are hereby incorporated by reference in their entireties.

This invention is directed to topically applied pharmaceutical products and methods for the transdermal, subdermal, transmembrane, musculoskeletal, transungual, transonychial, and/or peronychial delivery of an active ingredient to a patient. The topically applied pharmaceutical products of the invention comprise (a) an active ingredient, (b) an organogel, and (c) a non-irritating permeation enhancer. This invention is particularly directed to such topically applied pharmaceutical products for the enhanced delivery of a therapeutically effective amount of an active ingredient across keratinized tissues, such as skin, nails, and hair; across membranes below such keratinized tissues; across membranes of body cavities; and/or into body tissues and/or systems in proximity to any of the foregoing. This invention is more particularly directed to such topically applied pharmaceutical products for the enhanced delivery of a therapeutically effective amount of an active ingredient to a diseased or infected nail bed, nail matrix, and/or nail plate in the toenails and/or fingernails of a patient. More particularly still, the present invention is directed to such a topically applied pharmaceutical product for the enhanced delivery of a therapeutically effective amount of an active ingredient to a diseased or infected nail bed, nail matrix, and/or nail plate in the toenails or fingernails of a patient, wherein the disease or infection is onychomycosis.

Onychomycosis (tinea unguium) is a disease of the toenails and/or fingernails caused by an infection of the nail bed, nail matrix, and/or nail plate. The infection may be caused by dermatophyte (skin-, hair-, or nail-infecting) microorganisms that feed upon keratinized tissue such as the microoganisms Trichophyton rubrum, Trichophyton mentagrophytes, and Epidermpophyton floccosum; or may be caused by yeasts such as Candida albicans, Candida parapsiliosis, Candida tropicalis, or Torulopsis glabrata. Onychomycosis may also be caused by molds such as Scopulariopsis brevicaulus, or Scytalidium hyalinum. Onychomycosis may cause the toenails and/or fingernails to thicken, discolor, disfigure, and/or split. Although concern over onychomycosis is mainly cosmetic, the disease if left untreated may result in toenails becoming so thick that they press against the insides of shoes, causing pressure, irritation, and pain. Of still greater moment, however, are recent studies showing that diabetics infected with onychomycosis suffer a higher rate of amputation than do diabetics without the infection.

Onychomycosis is a particularly difficult fungal infection to treat systemically because nails grow slowly and receive very little blood supply; these challenges are amplified in the case of diabetics who commonly suffer poor circulation in the extremities. Onychomycosis can also be an expensive fungal infection to treat, since, in addition to requiring an extended (months-long) course of care, the few approved systemic (orally administered) treatments have side effects, most notably liver impairment that may require significant patient screening and/or monitoring. Currently, itraconazole (Sporanox® capsules) and terbinafine (Lamisil® tablets) are the preferred systemic (orally administered) therapies, but FDA has issued a Public Health Advisory warning that Sporanox® therapy may pose a risk of congestive heart failure, and that both Sporanox® and Lamisil® are associated with serious hepatic toxicity, including liver failure and death. (See FDA/Center for Drug Evaluation and Research, Last Updated: May 9, 2001 http://www.fda.gov/cder/drug/advisory/sporanox-lamisil/advisory.htm). Additionally, treating onychomycosis in the immunocompromised patient (such as those suffering from HIV) with systemic (e.g., orally administered) itraconazole may be ineffective and even dangerous. Itraconazole capsules require an acidic environment for absorption; patients suffering from HIV may have decreased acid secretion owing to secondary HIV gastropathy, whereby therapeutic concentrations of itraconazole might not be achievable. Futhermore, itraconazole is metabolised by the cytochrome p450 3A4 enzyme as are indinavir, lopinavir, and other HIV medications. The coadministration of oral itraconazole with such medications has significant potential for pharmacokinetic interaction and toxicity. (See Young E, Goldman M, Histoplasmosis and HIV Infection, HIV InSite Knowledge Base Chapter May 2005). There is therefore a need in the art for alternative therapeutic modalities for treating onychomycosis in the general population and a particular need in the art for alternative therapeutic modalites for treating onychomycosis in the diabetic and/or immunocompromised patient.

In addition to the above-noted shortcomings of current onychomycosis therapies, there are many known drawbacks associated with systemic treatments of infection, disease, or pain, through oral administrations and/or transcutaneous injections of medications, in general. Depending upon the medication and the metabolitic vicissitudes of any given patient, oral administration can respectively cause gastrointestinal irritations and variable rates of absorption, each of which can effect the efficacy of a treatment regimen through either the patient's understandable reluctance to comply therewith, or the achievement of less than therapeutic serum concentrations. For example, oral administration of non-steroidal anti-inflammatory drugs (NSAIDS) such as aspirin, ibuprofen, naproxen, and diclofenac can cause irritation or even ulceration of the lining of the stomach. The effective oral administration of other drugs is further hampered by litanies of other undesirable side effects, issues for pregnant or nursing women, and tendencies for interactions with co-administrations. The treatment of diseases and/or infections of either the toenails and/or fingernails presents a particular challenge to orally administered therapies since the toenails and fingernails generally receive less blood than other body tissues.

The transcutaneous injection of medications to treat infection, disease, or pain is similarly fraught with undesirable deficiencies. Aside from its obviously painful nature, transcutaneous injection often requires in-office administration by trained healthcare personnel, making it both more costly and more inconvenient for the patient. Additionally, despite modern cautions, and hightened hygienic conditions and practices, injections always present the possibility of causing infections in patients. Lastly, injection is frequently not location specific, but rather is administered at a location on the body that is remote from the site of infection, disease, or pain. Therefore, as with oral administration, transcutaneous injection of a medication results in transport losses, and necessitates that the patient receive more medication than would be necessarily therapeutic for treating the localized site of infection, disease, or pain.

The delivery of medications and therapeutic agents through keratinous tissues (e.g., skin, toenails, fingernails, and hair); or through membranes; by way of topically applied pharmaceutical compositions presents an advantageous alternative to therapies such as oral administration and trancutaneous injection. The transdermal, subdermal, transmembrane, musculoskeletal, transungual, transonychial, and/or peronychial delivery of medications and therapeutic agents through topically applied compositions avoids gastrointestinal complications, nullifies the influence of patient metabolism, and greatly reduces the risk of untoward side effects and/or interactions with coadministrations. However, the transdermal, subdermal, transmembrane, musculoskeletal, transungual, transonychial, and/or peronychial delivery of medications and therapeutic agents through topically applied compositions presents two significant challenges. Foremost among these challenges is the fact that skin, nails, and membranes are defensive barriers designed to keep foreign pathogens and toxic substances from entering the body, while simultaneously preventing physiological fluids and nutrients from leaving the body. Accordingly, the stratum corneum (the outer visible layer of the skin derived from the epidermis) is a horny, abrasion-resistant layer composed of thinly-stacked, keratin-filled dead denucleated cells called keratinocytes. This dead cell matrix of the stratum corneum is lipid-rich, providing the body with a water-resistant and highly impermeable barrier to the outside world. The nail, similarly derived from the epidermis, is composed mostly of highly disulfide-linked keratin and is approximately 100 times thicker than the stratum corneum, thereby providing an even more daunting barrier to medication delivery. Additionally, many of the various membranes of the body are very selectively permeable, if permeable at all. The limited permeabilities of these various defensive barriers and the extent to which those limited permeabilities will hinder the delivery of a therapeutically effective amount of a medication must be considered in formulating any topically applied pharmaceutical composition.

One way of increasing a drug's delivery through such tissues (and into underlying tissues and/or systems, if additionally desired) is to use a permeation enhancer (alternatively referred to in the art and herein as a penetration enhancer). However, the second serious challenge to the formulation of a topically applied pharmaceutical composition is ensuring that it is biocompatible with the tissues to which it is to be applied (i.e., skin, nails, hair, and membranes) such that the tissues will not be damaged or irritated by the formulation, or by its components. This is particularly challenging since many topical excipients that are known to be permeation enhancers are also known to be skin and membrane irritants.

Because they are biocompatible with skin, nails, and hair; able to solubilize drugs; and believed capable of modifying keratinous tissues to provide desired drug partitioning, there is significant interest in organogels as potential carrier vehicles for the topical delivery of drugs. Likely owing to the interaction of their phospholipid components with the biolipids of keratinous tissues (e.g., the stratum corneum) organogels are able to deliver active ingredients across keratinous barriers faster than they could be delivered across such barriers without an organogel carrier.

Organogels are jelly-like compositions generally synthesized by adding a polar solvent to an organic solution of a phospholipid, such as lecithin—commercially derived from soybean. Lecithin dissolved in organic media self-assembles into reverse spherical micelles (e.g., like drops of water in oil). Upon the addition of a polar solvent, such as water, the hydrophilic heads of the lecithin molecules bind stoichiometrically, such that adjacent lecithin molecules are bridged by one polar molecule. Hydrogen bonding between the polar solvent and the phosphate groups of the lecithin molecules gives rise to the formation of linear networks. Further additions of polar solvent result in the formation of long tubular micelles that may be hundreds of thousands of nanometers long. Upon reaching a critical length these giant, extended, spaghetti-like micelles overlap and entangle themselves into a three-dimensional network that results in an organogel of high organization and macroscopic viscosity. These organogels may comprise as much as 85% by weight of external phase: the external phase being the organic solvent that is immobilized and entrapped in the spaces between the entangled reverse micelles.

A limitation of earlier organogel formulation practice was the then-perceived need to use highly pure lecithin, which was expensive and not easily obtained. However, more recent formulation practice has incorporated synthetic polymers known as “pluronics,” which have facilitated gelation with lecithin of lesser purity. These pluronics are block copolymers of poly(ethylene-oxide) and poly(propylene-oxide), and are often referred to as poloxamers or poloxamer polyols. Pluronics are known in the pharmaceutical arts for use as co-surfactants, gelling agents, emulsifying agents, solubilizers, suspending agents, and stabilizers. Lecithin organogels utilizing pluronics are often referred to as “pluronic lecithin organogels,” “poloxamer oganogels,” “pluronic organogels,” “pluronic lecithin liposomal organogels” or simply “PLOs.” (See each of U.S. Pat. Nos. 5,976,547; 5,945,409; and 5,837,289).

As previously stated, a particular challenge in formulating topical compositions for transdermal, subdermal, transmembrane, musculoskeletal, transungual, transonychial, and/or peronychial delivery of an active ingredient to a patient is avoiding damage or irritation to the skin, nails, hair, or membranes to which the topical composition is to be applied. While organogels themselves are known to be biocompatible, the limited number of permeation (penetration) enhancers with which the art has paired them are not so benign.

U.S. Pat. No. 5,976,547 issued to Archer et al. discloses a topical analgesic and antiphlogistic composition comprising: an active agent (such as the NSAID ketoprofen) blended in either a pluronic lecithin organogel (PLO) or a petrolatum base, along with the herbal extract arnica montana as a penetration enhancer. However, the adverse effects associated with this notably impure herbal extract may militate against its continued use in such topical formulations. Spettoli et al. have reported positive reactions for contact dermatitis in a patient treated with arnica tinture (obtained from arnica montana) in 20% petrolatum. (See Spetolli E, Slivani S, Lucente P, Guerra L, Vincenzi C; Contact dermatitis caused by sesquiterpene lactones; Am J Contact Dermat. 1998 Mar; 9(l):49-50). Paulsen also noted that arnica montana has been suspected of sensitization or elicitation of Compositae dermatitis, and that, based on epidemiological data, sensitization seems to occur relatively frequently with the use of this species of herbal extract. (See Paulsen E; Contact sensitization from Compositae-containing herbal remedies and cosmetics, Contact Dermatits. 2002 October; 47(4): 189-98).

U.S. Pat. No. 6,914,051 issued to Allen discloses a topical gel for treating pain, inflammation, and other pathological conditions affecting musculoskelatal tissues and other soft tissues of the body. The topical gel comprises an antibiotic, a pluronic lecithin liposomal organogel (PLO), and optionally a penetration enhancer. The preferred penetration enhancer is d-limonene (a terpene) which is known in the art to be a skin and eye irritant. (See International Programme on Chemical Safety, Concise Int'l Chemical Assessment Doc No. 5; World Health Organization, Geneva, 1998). (See also The Merck Index 12^{th} Edition, page 938, noting limonene as a “skin irritant, sensitizer”). The several other penetration enhancers mentioned in the patent are “various other terpenes,” which as a class are known to cause contact dermatitis, and “decyl-methyl sulfoxid[e] and DMSO,” which are also known in the art to be irritating to the skin and/or other body tissues. (See U.S. Pat. No. 5,028,431 listing decyl methyl sulfoxide amongst a litany of “irritating enhancers”). (See also The Merck Index 12^{th} Edition, page 551, attributing to DMSO “primary irritation with redness, itching and sometimes scaling”).

U.S. Pat. No. 5,837,289 issued to Grasela et al. discloses a topical cream for transdermal delivery of a medication. The cream is comprised of a solubilized or intimately suspended medication, an organogel containing a non-irritating penetration enhancer, and a polymeric component. Although the specification notes that the components of the cream “must not be toxic, irritating or otherwise harmful to the patient[,]” and that the enhancers in particular should “have no irritancy and toxicity to the skin, and the whole body[,]” in preferred embodiments the penetration enhancer in the organogel is either isopropyl palmitate or isopropyl myristate. This preference is surprising since both isopropyl palmitate and isopropyl myristate are known skin irritants that can cause allergic reactions and aggravate acne conditions. Indeed, U.S. Pat. No. 5,945,409 to Crandall notes that “IPP and IPM can be irritating to sensitive facial skin and may produce comedowns” [sic, comedones] (i.e., acne pimples).

Willimann et al. have studied the use of lecithin organogels containing organic solvents as matrices for the transdermal transport of drugs, among them scopolamine (an anti-motion sickness agent) and broxaterol (a bronchiodilating agent). (See Willimann H, Walde P, Luisi P L, Gazzaniga A, Stroppolo F; Lecithin Organogel as Matrix for Transdermal Transport of Drugs; J Pharm Sci. 1992; 81:871-874). The organic solvents used by this team were the aforementioned isopropyl palmitate (IPP), and cyclooctane. Drug solubility was noted to be increased in lecithin-IPP solution compared to drug solubility in IPP alone, evincing the drug solubility enhancing properties of the organogel. Additionally, the IPP-based lecithin gel demonstrated better transdermal transport efficiency than that of the cyclooctane-based lecithin gel, suggesting better penetration enhancing properties for IPP over cyclooctane. Although Willimann et al. reported no significant alterations to skin after three days of application with either the isopropyl palmitate solvent alone, or with a lecithin-isopropyl palmitate gel, these were only preliminary histological, in vitro studies. Moreover Willimann et al. offered no similar skin-effect study with either the cyclooctane solvent alone, or with any lecithin-cyclooctane gel. While the art-recognized deleterious effects that IPP may have on skin have already been noted [production of acne pimples (comedones)], the art further recognizes that exposure to cyclooctane may result in irritation to the eyes, skin, and respiratory systems. (See CDN Isotopes Inc., Material Safety Data Sheet No. 3440, signed May 8, 2004). Indeed, Willimann itself notes that “cyclooctane is not interesting for physiological application[.]” Accordingly, there is a need in the art for topically applied pharmaceutical compositions and methods that allow for the therapeutically effective transdermal, subdermal, transmembrane, mucoskeletal, transungual, transonychial, and/or peronychial delivery of an active ingredient to a patient, which do not use permeation enhancers that irritate or damage the tissues to which such compositions are applied, or by which they are ultimately absorbed.

Drastically underscoring the particular challenge that medicinal delivery through the toenails and fingernails (transungual, transonychial, or peronychial delivery) continues to pose to the art, especially in the treatment of onychomycosis, is recent U.S. Patent Application Publication 20040197280, published in the name of Repka. Repka's method of medicinal delivery does not use an organogel, but rather uses a chemical or mechanical etching process, and optionally a penetration enhancer. The chemical and mechanical etching processes conceive of any of “acids,” “a laser,” “a mini-sandblaster,” or “a nail file,” with which microporosities are created within the nail surface, thereby increasing surface area and wettability. Repka teaches that his method is a preferable alternative to nail avulsion (removal of the nail), which he notes as being a prerequisite to “effective” topical antifungal treatment of onychomycosis. Repka's preferred penetration enhancers (urea, sodium sulfide, and ammonium thioglycolate) may yet better illustrate the pressing need for development of practical alternative therapeutic modalities for the treatment of onychomycosis than does his use of acids, lasers, or mini-sandblasters in preference to nail avulsion. Sodium sulfide (CAS Number: 1313-82-2) is known to be a strong irritant that can cause caustic burns and painful inflammation to skin, eyes, and mucous membranes. The Merck Index, in addition to noting its use in “dehairing hydes and wool pulling,” warns that upon exposure to air sodium sulfide produces hydrogen sulfide, and that it should not be handled with bare hands. (See The Merck Index 12^{th} Edition, page 1483). Urea (CAS Number: 57-13-6) is also a well known skin irritant and laboratory hazard. (See Taylor A, Feller S, Structural Studies of Phycobiliproteins from Spirulina: Combining Spectroscopy, Thermodynaics, and Molecular Modeling in an Undergraduate Biochemistry Experiment; J Chem Ed. Vol. 790, No. 12 December 2002). Similarly, ammonium thioglycolate (CAS Number: 5421-46-5) is known to cause irritant contact dermatitis in hairdressers and their clients. (See Haz-Map; U.S. Library of Medicine, National Institutes of Health @hazmap.nlm.nih.gov). Accordingly, there is a particular need in the art for practical, topically applied pharmaceutical compositions and methods that allow for the therapeutically effective transungual, transonychial, and/or peronychial delivery of an active ingredient to a patient to treat onychomycosis, which do not use penetration enhancers that irritate or damage the nails or surrounding tissues to which such compositions are applied, or by which they are ultimately absorbed.

Addressing the above-noted needs of the art, the instant invention relates generally to a topically applied pharmaceutical composition, as well as to the use and formulation thereof.

More specifically this invention relates to topically applied pharmaceutical products and methods for the transdermal, subdermal, transmembrane, musculoskeletal, transungual, transonychial, and/or peronychial delivery of an active ingredient to a patient.

In accordance with one aspect of the invention, there is provided a topically applicable pharmaceutical product comprising (a) an active ingredient, (b) an organogel, and (c) a non-irritating permeation enhancer.

In a preferred embodiment the organogel component is lecithin-based and contains a pluronic-type surfactant, being thus a “Pluronic Organogel,” or “PLO,” as those terms are generally known in the art.

In a preferred embodiment the non-irritating permeation enhancer component is a macrocyclic compound of the “Hsieh” type, such as are disclosed in U.S. Pat. No. 5,023,252 and U.S. Pat. No. 5,731,303, each of which Patents is hereby incorporated herein by this reference in its entirety.

Thus, there is provided a topically applicable composition, which by virtue of its organogel component, has a specific molecular structure that enables it to deliver increased amounts of molecules through biological membranes, such as skin and mucous layers. Organogels have been demonstrated to have a unique molecular matrix, structured so as to simulate the composition and molecular structure of the skin; this has been shown to aid the absorption of a drug dispersed or dissolved in the matrix.

The composition of the present invention is novel, however, in that the organogel's mechanism of delivery, that of emulating the skin structure, is additionally augmented by the presence of the macrocyclic compound (non-irritating permeation enhancer), which is known to also promote drug absorption across the skin without irritation or damage thereto. This combination of two independent delivery mechanisms confers the additional benefit of increasing the absorption of molecules, especially drug molecules desirable for transdermal, subdermal, transmembrane, musculoskeletal, transungual, transonychial, peronychial, or systemic delivery, beyond that achievable by either the organogel structure, or the absorption promoter (non-irritating permeation enhancer) acting alone. It is a further and particularly important advantage of the present composition that the increased absorption may be achieved without irritating or damaging the tissues to which the composition is topically applied, or any further body tissue to which the active ingredient is intended to be ultimately delivered.

This invention may be used to deliver any active ingredient including medicinal agents, pharmaceutical compounds, drugs, or cosmetic agents that are desirably administered topically to the skin, nails, hair, and/or membranes for transdermal, subdermal, transmembrane, musculoskeletal, transungual, transonychial, and/or peronychial treatment or regional treatment.

As used herein the term “active ingredient” shall mean any organic or inorganic compound or substance having bioactivity and/or which is adapted for, and/or used for, a therapeutic purpose. As non-limiting examples of active ingredients that are useful in the topically applied pharmaceutical products and methods of the instant invention there may be mentioned: antifungal agents; anti-inflammatory agents, such as non-steroidal anti-inflammatory drugs (NSAIDS) and steroidal anti-inflammatory drugs; antibiotics; antiviral agents; anti-neoplastic agents; astringents; anesthetics; systemic drugs; steroid hormones, such as estradiol and testosterone; cosmetic agents, such as skin moisturizers, protectants, and emollients; nutrients, such as vitamins; and ceramides.

As non-limiting examples of antifungal agents that can be employed in the composition of the invention there may be mentioned: amphotericin B, flucytosine, fluconazole, griseofulvin, miconazole nitrate, terbinafine hydrochloride, ketoconazole, itraconazole, undecylenic acid and chloroxylenol, ciclopirox, clotrimazole, butenafine hydrochloride, nystatin, naftifine hydrochloride, oxiconazole nitrate, selenium sulfide, econazole nitrate, terconazole, butoconazole nitrate, carbol-fuchsin, clioquinol, methylrosaniline chloride, sodium thiosulfate, sulconazole nitrate, tioconazole, tolnaftate, undecylenic acid, and undecylenate salts (such as calcium undecylenate, copper undecylenate, and zinc undecylenate).

As non-limiting examples of anti-inflammatory drugs that can be employed in the composition of the invention there may be mentioned: aspirin; ibuprofen; naproxen; diclofenac; ketoprofen; flubiprofen; and the so-called “Cox-2” anti-inflammatory agents such as rofecoxib, celecoxib, etoricoxib, valdecoxib, lumiracoxib and other NSAIDs that are identified as being cyclooxygenase Type 2 inhibitors.

As non-limiting examples of antibiotics that can be employed in the composition of the invention there may be mentioned: cefadroxil, cefazolin, cephalexin, cephalothin, cephapirin, cephacelor, cephprozil, cephadrine, cefamandole, cefonicid, ceforanide, cefuroxime, cefixime, cefoperazone, cefotaxime, cefpodoxime, ceftaxidime, ceftibuten, ceflizoxime, ceftriaxone, cefepime, cefinetazole, cefotetan, cefoxitin, loracarbef, imipenem, erythromycin (and erythromycin salts such as estolate, ethylsuccinate, gluceptate, lactobionate, stearate), azithromycin, clarithromycoin, dirithromycin, troleanomycin, penicillin V, penicillin salts, and complexes, methicillin, nafcillin, oxacillin, cloxacillin, dicloxacillin, amoxicillin, amoxicillin and clavulanate potassium, ampicillin, bacampicillin, carbenicillin indanyl sodium (and other salts of carbenicillin) mezlocillin, piperacillin, piperacillin and taxobactam, ticarcillin, ticarcillin and clavulanate potassium, clindamycin, vancomycin, novobiocin, aminosalicylic acid, capreomycin, cycloserine, ethambutol HCl and other salts, ethionamide, and isoniazid, ciprofloxacin, levofloxacin, lomefloxacin, nalidixic acid, norfloxacin, ofloxacin, sparfloxacin, sulfacytine, suflamerazine, sulfamethazine, sulfamethixole, sulfasalazine, sulfisoxazole, sulfapyrizine, sulfadiazine, sulfmethoxazole, sulfapyridine, metronidazole, methenamine, fosfomycin, nitrofurantoin, trimethoprim, clofazimine, co-triamoxazole, pentamidine, and trimetrexate.

As non-limiting examples of antiviral agents that can be employed in the composition of the invention there may be mentioned: Acyclovir, Amantadine, Amprenavir, Cidofovir, Delavirdine, Didanosine, Famciclovir, Foscamet, Ganciclovir, Indinavir, Interferon, Lamivudine, Nelfinavir, Nevirapine, Palivizumab, Penciclovir, Ribavirin, Rimantadine, Ritonavir, Saquinavir, Stavudine, Trifluridine, Valacyclovir, Vidarabine, Zalcitabine, and Zidovudine.

As non-limiting examples of anti-neoplastic agents that can be employed in the composition of the invention there may be mentioned: carboplatin, busulfan, cisplatin, thiotepa, melphalan hydrochloride, cyclophosphamide, ifosfamide, chlorambucil, mechlorethamine hydrochloride, carmustine, lomustine, streptozocin, polifeprosan 20, dexrazoxane, dronabinol, granisetron hydrochloride, fluconazole, erythropoietin, octreotide acetate, pilocarpine hydrochloride, etidronate disodium, pamidronate disodium, allopurinol sodium, amifostine, filgrastim, mesna, ondansetron hydrochloride, dolasetron mesylate, leucovorin calcium, sargramostim, levamisole hydrochloride, doxorubicin hydrochloride, idarubicin hydrochloride, mitomycin, daunorubicin citrate, plicamycin, daunorubicin hydrochloride, bleomycin sulfate, mitoxantrone hydrochloride, valrubicin, dactinomycin, fludarabine phosphate, cytarabine, mercaptopurine, thioguanine, methotrexate sodium, cladribine, floxuridine, capecitabine, anastrozole, bicalutamide, tamoxifen citrate, testolactone, nilutamide, methyltestosterone, flutamide, toremifene citrate, goserelin acetate, estramustine phosphate sodium, ethinyl estradiol, esterified estrogen, leuprolide acetate, conjugated estrogens, megestrol acetate, aldesleukin, medroxyprogesterone acetate, dacarbazine, hydroxyurea, etoposide phosphate, megestrol acetate, paclitaxel, etoposide, teniposide, trastuzumab, rituximab, vinorelbine tartrate, denileukin diftitox, gemcitabine hydrochloride, vincristine sulfate, vinblastine sulfate, asparaginase, edrophonium chloride, bacillus calmette and guerin, irinotecan hydrochloride, pegaspargase, docetaxel, interferon alfa-2a, recombinant, tretinoin, porfimer sodium, interferon alfa-2b, recombinant, procarbazine hydrochloride, topotecan hydrochloride, altretamine, fluorouracil, prednisolone sodium phosphate, cortisone acetate, dexamethasone, dexamethasone sodium sulfate, dexamethasone acetate, hydrocortisone sodium phosphate, hydrocortisone, prednisolone, methylprednisolone sodium succinate, betamethasone sodium phosphate, betamethasone acetate, letrozole, mithramycin, mitotane, pentostatin, perfosfamide, and raloxifene.

In a preferred embodiment the active ingredient is an antifungal agent. In a more preferred embodiment the active ingredient is an antifungal agent that is suitable for treating onychomycosis. In a particularly preferred embodiment the active ingredient is terbinafine.

In general, the topically applicable pharmaceutical products and methods of the invention are formulated to so as to enhance the delivery of a therapeutically effective amount of an active ingredient across keratinized tissues, such as skin, nails, and hair; across membranes below such keratinized tissues; across membranes of body cavities; and/or into body tissues or systems in proximity to any of the foregoing.

As used hereinabove and hereinbelow and as understood in the relevant art's broadest interpretation thereof, the term “therapeutically effective amount” shall mean an amount, dose regimen, or treatment protocol, or combination thereof, that achieves a successful treatment effect.

As used hereinabove and hereinbelow and as understood in the relevant art's broadest interpretation thereof, the term “transdermal” shall mean through the skin.

As used hereinabove and hereinbelow and as understood in the relevant art's broadest interpretation thereof, the term “subdermal” shall mean below the skin.

As used hereinabove and hereinbelow and as understood in the relevant art's broadest interpretation thereof, the term “transmembrane” shall mean through a membrane.

As used hereinabove and hereinbelow and as understood in the relevant art's broadest interpretation thereof, the term “musculoskeletal” shall mean pertaining to the muscles and/or bones.

As used hereinabove and hereinbelow and as understood in the relevant art's broadest interpretation thereof, the terms “transonychial” or “transungual” shall mean through the nail.

As used hereinabove and hereinbelow and as understood in the relevant art's broadest interpretation thereof, the term “peronychial” shall mean any, or all, of through the peronychium; or through any tissue surrounding, bordering, or otherwise communicating with a fingernail or toenail.

In accordance with an aspect of the invention the topically applied pharmaceutical compositions allow for the enhanced transdermal, subdermal, transmembrane, musculoskeletal, transungual, transonychial, and/or peronychial delivery of a therapeutically effective amount of an active ingredient to a patient.

Notwithstanding any description hereinabove or hereinbelow to a delivery of a therapeutically effective amount of an active ingredient to a patient as being any of transdermal, subdermal, transmembrane, musculoskeletal, transungual, transonychial, and/or peronychial, such description shall not mean that such delivery is limited to such description. Accordingly, the topically applicable pharmaceutical products and methods of the invention may also be formulated to so as to enhance the delivery of a therapeutically effective amount of an active ingredient across any of the aforementioned tissues and membranes and into further body tissues. As non-limiting examples of further body tissues to which such active ingredients may be delivered by way of the instant invention there may be mentioned: nail bed, nail matrix, nail plate, muscles, glands, organs, bones, joints, circulatory systems, central nervous systems, endocrine systems, and lymph nodes.

In accordance with one aspect of the instant invention, the topically applied pharmaceutical composition is intended and designed to treat a patient in need of treatment for, or in need of prevention of, various indications generally including but not limited to: infection, disease, and/or pain. As non-limiting examples of more specific indications that the topically applied pharmaceutical compositions of the instant invention may be used to treat, or to prevent, there may be mentioned: onychomycosis; skin fungal infections, such as athletes' foot; dermatoses; psoriasis; sun bum; and chemical dependencies, such as smoking and alcoholism.

In accordance with a further aspect of the instant invention the composition is intended and designed to enhance delivery of a therapeutically effective amount of an active ingredient to a diseased or infected nail bed, nail matrix, and/or nail plate in the toenails and/or fingernails of a patient.

In accordance with a still further aspect of the instant invention the composition is intended and designed to enhance delivery of a therapeutically effective amount of an active ingredient to a diseased or infected nail bed, nail matrix, and/or nail plate in the toenails or fingernails of a patient, wherein the disease or infection is onychomycosis.

The permeation enhancing components used in the instant invention are selected from the class of “Hsieh-type” macrocyclic enhancers and preferentially are the ones that have known regulatory status and/or are known to be the non-irritating ones. The Hsieh-type permeation enhancers used in the present invention comprise those of this class that not only are non-irritating and/or of beneficial regulatory status, but that also confer useful properties to the formulation. The composition of the present invention is a new construct, which in addition to comprising an active ingredient and an organogel, further comprises a non-irritating permeation enhancer (specifically a Hsieh type enhancer) that additionally facilitates drug transport to the skin. These non-irritating, non-damaging, biocompatible, Hsieh-type enhancers actually facilitate the hydration of the skin, thereby also conferring skin protectant properties.

The medicinal organogel so produced is compatible with skin, nails, hair, and membranes.

The instant invention is an advantageous alternative to delivery of an active ingredient by transcutaneous injection.

U.S. Pat. No. 5,023,252 describes a composition for delivery of an active ingredient, particularly insulin, by a route other than by transcutaneous injection. More particularly, such patent describes the use of compositions that include permeation enhancers for delivery of active ingredient through skin and membranes of body cavities without requiring an injection.

The present invention is directed to new topically applied organogel compositions that contain such permeation enhancers and the use thereof.

In accordance with the invention, there is provided a pharmaceutical composition comprising: (a) an active ingredient, (b) an organogel, and (c) a non-irritating permeation enhancer.

Applicant has found that when using a composition that contains a combination of (a) an active ingredient, (b) an organogel, and (c) a non-irritating permeation enhancer improved results are obtained when the composition is at an acidic pH.

The invention further relates to treating a patient in need of an active ingredient with a combination of (a) an active ingredient, (b) an organogel, and (c) a non-irritating permeation enhancer; the combination having an acidic pH of no greater than 4.5. Preferably the pH of the composition is no greater than 4 nor below 2. The pH is preferably at least 3.

In general, the pH of the composition is at least 2 and no greater than 4.5. In a preferred embodiment, the pH is no greater than 4. A preferred range of pH is from 2.5 to 3.8. In one preferred embodiment the pH is about 3 to 3.5.

The pH of the composition may be maintained by the use of a suitable buffer. The selection of a buffer to maintain the desired pH is deemed to be within the scope of those skilled in the art based on the teachings set forth herein. As representative examples of suitable buffers there may be mentioned citric acid buffer, phosphate buffer, and the like, as is in common use and also suitable for medical formulations.

In general, the non-irritating permeation enhancer that is employed is one that enhances the permeation of the active ingredient through a patient's skin, nails, hair, and body cavity membranes.

In general, the non-irritating permeation enhancer that is employed is one that enhances the permeation of the active ingredient through any of a patient's skin, nails, hair, membranes, and further body tissues, and in particular is one that enhances the permeation of the active ingredient through a patient's toenails or fingernails, to the nail bed, nail matrix, and/or nail plate.

In a composition containing an effective amount of active ingredient a preferred non-irritating permeation enhancer is a compound of the structure:

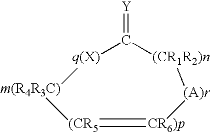

wherein X and Y are oxygen, sulfur or an imino group of the structure

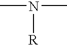

or ═N—R with the proviso that when Y is the imino group, X is an imino group, and when Y is sulfur, X is sulfur or an imino group, A is a group having the structure

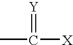

wherein X and Y are defined above, m and n are integers having a value from 1 to 20 and the sum of m+n is not greater than 25, p is an integer having a value of 0 or 1, q is an integer having a value of 0 or 1, r is an integer having a value of 0 or 1, and each of R, R₁, R₂, R₃, R₄, R₅ and R₆ is independently hydrogen or an alkyl group having from 1 to 6 carbon atoms which may be straight chained or branched provided that only one of R₁ to R₆ can be an alkyl group, with the proviso that when p, q and r have a value of 0 and Y is oxygen, m+n is at least 11, and with the further proviso that when X is an imino group, q is equal to 1, Y is oxygen, and p and r are 0, then m+n is at least 11, and said compound will enhance the rate of the passage of the drug across skin, nails, hair, and body cavity membranes. Hereinafter these compounds are referred to as enhancers. When R, R₁, R₂, R₃, R₄, R₅ or R₆ is alkyl it may be methyl, ethyl, propyl, isopropyl, butyl, isobutyl, sec-butyl, amyl, hexyl, and the like. Such permeation enhancers are described in U.S. Pat. No. 5,023,252 and U.S. Pat. No. 5,731,303.

Preferably, the enhancer compounds used in this invention are the cyclic lactones (the compounds wherein both X and Y are oxygen, (q is 1 and r is 0), the cyclic diesters (the compounds wherein both X and Y are oxygen, and both q and r are 1), and the cyclic ketones (the compounds wherein both q and r are 0 and Y is oxygen). In the cyclic diesters m+n is preferably at least 3. In the cyclic ketones m+n is preferably from 11 to 15 and p is preferably 0.

Enhancers of the above structural formula may be referred to herein as “Hsieh-type” enhancers and are described, for example, in aforementioned U.S. Pat. Nos. 5,023,252 and 5,731,303 (“Hsieh Patents”). Such enhancers are lipophilic and are “compatible” with skin, nails, hair, and body cavity membranes meaning that they do not cause damage to the skin, nails, hair, or body cavity membranes on which the composition of the present invention is to be applied (hereinafter “target surface”). Such enhancers produce either a low level of irritability or no irritability to the target surface and, in fact serve as an emollient.

Preferred enhancers for use in the present invention are macrocyclic enhancers. The term “macrocyclic” is used herein to refer to cyclic compounds having at least 12 carbons in the ring. Examples of preferred macrocyclic enhancers for use in the present invention include: (A) macrocyclic ketones, for example, 3 methylcyclopentadecanone (muscone), 9-cycloheptadecen-1-one (civetone), cyclohexadecanone, and cyclopentadecanone (normuscone); and (B) macrocyclic esters, for example, pentadecalactones such as oxacyclohexadecan-2-one (cyclopentadecanolide, ω-pentadecalactone).

Oxacyclohexadecan-2-one and cyclopentadecanone are especially preferred.

Although the above are preferred permeation enhancers, one of ordinary skill in the art would recognize that the instant teachings would also be applicable to other permeation enhancers. Non-limiting examples of other permeation enhancers useful in the instant invention are the simple long chain esters that are Generally Recognized As Safe (GRAS) in the various pharmacopoeial compendia. These may include simple aliphatic, unsaturated or saturated (but preferably fully saturated) esters, which contain up to medium length chains. Non-limiting examples of such esters include myristyl myristate, octyl palmitate, and the like. The enhancers are of a type that are suitable for use in a pharmaceutical composition. The artisan of ordinary skill will also appreciate that those materials that are incompatible with skin, nails, hair, body cavity membranes, and/or mucous membranes, or which are irritating to skin, nails, hair, body cavity membranes, and/or mucous membranes should be avoided, such as arnica montana, d-limonene, terpenes, decyl-methy sulfoxid[e], DMSO, isopropyl myristate, isopropyl palmitate, cycoloctane, urea, sodium sulfide, ammonium thioglycolate, and pungent oils, such as Tea tree oil and Eucalyptus oil.

The enhancer is present in the composition in a concentration effective to enhance penetration of the active ingredient through the target surface, when the composition is topically applied to the target surface to effect transdermal, transmembrane, transungual, transonychial, or peronychial delivery of the active ingredient. Various considerations should be taken into account in determining the amount of enhancer to use. Such considerations include, for example, the amount of flux (rate of passage through the target surface) achieved and the stability and compatibility of the components in the formulations. The enhancer is generally used in an amount of about 0.1 to about 25 wt. % of the composition, more generally in an amount of about 1 to about 15 wt. % of the composition, and in preferred embodiments in an amount of about 0.5 to about 15 wt. % of the composition.

The organogel is present in the composition in a concentration effective to serve as a suitable vehicle for the compositions of the present invention. In general, the organogel is used in an amount of about 40 to about 99 wt. % of the composition and in preferred embodiments in an amount of about 80 to about 98 wt. % of the composition.

The organogel composition of the present invention is preferably in the form of a cream, but it may also be in the form of lacquer, gel, foam, salve, ointment, lotion, solution, or other form suitable for topical application, as are known in the art. In an embodiment, the preferred organogel is a Pluronic organogel with the active ingredient being dispersed or dissolved in the organogel in a therapeutically effective amount.

Generally, these are structured creams that are self-emulsifying when mixed by conventional mixing means, such as by a Hobart mixer or by paddle mixer. In one preferred embodiment, the permeation enhancer is mixed in the organogel that contains the active ingredient. Further emulsification, if needed, may be effected through the use of one or more suitable surfactants. The selection of a suitable surfactant is deemed to be within the scope of those skilled in the art based on the teachings herein. Essentially any suitable surfactant or mixture of surfactants can be used in the practice of the present invention, including, for example, anionic, cationic, and non-ionic surfactants. Preferred surfactants are non-ionic surfactants, with those having a hydrophilic-lipophilic balance (HLB) of from about 7 to about 14 being particularly preferred. Examples of such non-ionic surfactants are PEG-60 corn glycerides, PEG-20 sorbitan monostearate, phenoxy-poly(ethyleneoxy)ethanol, sorbitan monooleate, and the like. Especially preferred are compendial surfactants such as those described in compendia such as the Food Chemicals Codex, National Formulary, U.S. Pharmacopeia, and the Code of Federal Regulations. It is preferred that the resultant creams have a good ‘feel,’ such as being emollient and having a hydrating effect. For this result, modifiers may be used to impart emolliency. Particularly effective is the addition of suitable oils, such as cottonseed, soybean, palm, or olive oil in an amount sufficient to provide the desired effect, as known to persons of ordinary skill in the formulation art. About 1-5% of the above-mentioned oil has been shown to be effective, although these amounts are not meant to limit the invention.

In some embodiments the composition of the invention may contain an active ingredient that requires refrigeration, and such refrigeration may result in crystallization of the permeation enhancer. In order to inhibit or prevent such crystallization, in a preferred embodiment the composition includes one or more crystallization inhibitors to inhibit the crystallization of the permeation enhancer. Crystallization, if allowed to proceed, may render the cream unstable and has an adverse effect on shelf life. Preferred crystallization inhibitors function by lowering the temperature at which the involved compound crystallizes. Examples of such crystallization inhibitors include natural oils, oily substances, waxes, esters, and hydrocarbons. Examples of natural oils or oily substances include Vitamin E acetate, octyl palmitate, sesame oil, soybean oil, safflower oil, avocado oil, palm oil, and cottonseed oil. The selection of a suitable crystallization inhibitor is deemed to be within the scope of those skilled in the art from the teachings herein. Preferred crystallization inhibitors function by lowering the temperature at which the permeation enhancer crystallizes.

Inhibitors which are capable of lowering the temperature of crystallization of the involved compound to below about 25° C. are particularly preferred, with those capable of lowering the crystallization of the involved compound to below about 5° C. being especially preferred. Examples of especially preferred crystallization inhibitors for use in inhibiting the crystallization of oxacyclohexadecan-2-one include hexadecane, octyl palmitate, cottonseed oil, safflower oil, and Vitamin E acetate, each of which may be used in pharmaceutical preparations.

The crystallization inhibitor is present in the composition in a concentration effective to inhibit the crystallization of the permeation enhancer. In general the crystallization inhibitor is present in an amount of about 0.1 to about 5 wt. % of the composition, more generally in an amount of from about 0.5 to about 2 wt. % of the composition. In one embodiment the crystallization inhibitor is present in an amount of from about 1 to about 2 wt. % of the composition. The crystallization inhibitor is one preferably used when the enhancer has a crystallization temperature above about 0 degrees Centigrade. In particular, for example, a crystallization inhibitor is preferably used when the enhancer is, pentadecalactone and/or cyclohexadecanone, since these crystallize above room temperature.

The topically applied composition of the invention is generally employed in a dosing regimen that is dependent on the patient being treated. Thus the frequency of the use and the amount of the dose may vary from patient to patient. In general, dosing is in an amount of from about 1/20 to ⅕ the effective oral dose in milligrams for each toenail involved and the frequency of dose is 1 to 2 times per day. In a preferred embodiment the compositions and methods are directed to the treatment of onychomycosis and the dosage is generally from about 10 to 100 milligrams of a 3% active antifungal cream per involved toenail, more preferably about 50 to 100 milligrams. However, as is known in the art, the effectiveness of the treatment of a disease may vary from patient to patient, and based on known therapies and the teachings herein one skilled in the art can select the dosing regimen and dosage for a particular patient or patients.

In a preferred embodiment the composition of the present invention comprises terbinafine. The terbinafine is present in the composition in a therapeutically-effective amount. In general the terbinafine is present in an amount of about 0.01 to about 15 wt. of the composition, more generally an amount of about 1 to about 5 wt. %of the composition. In one embodiment the terbinafine is present in an amount of about 1 to about 3 wt. % of the composition.

In contrast to all of the permeation enhancers discussed in the hereinabove noted prior art, the permeation enhancer cyclopentadecanolide, as used in the preferred embodiments of the instant invention, is not only non-irritating to the skin, but has the property of being a hydrating agent, thereby imparting beneficial effects on the skin. Cyclopentadecanolide is currently listed by the FDA as an inactive pharmaceutical excipient and is used as a fragrance in sensitive skin soaps and underarm deodorants.

In a preferred embodiment the present invention provides a topically applicable composition in the form of a convenient, elegant, emollient cream.

It is an advantage of the present invention that it is easily applied to the target surface without the need of a brush or an applicator.

It is a further advantage of the present invention that no film former component is necessary, and hence no need for clean up with acetone or other nail polish removing chemicals.

It is a further advantage of the instant organogel/enhancer combination that it is a thicker cream at room temperature than organogels known in the art, and it therefore does not as readily undergo syneresis, i.e., the exudation of the liquid component from the cream (gel, lotion, foam) causing it to coagulate. Syneresis (or sweating) can adversely affect formula stability and the aesthetic properties of the cream when water or other solvents are exuded from the cream taking active ingredients with them; this is especially so when such products are shipped or stored in warm climates and/or high altitudes.

Though preferably in the form of a cream, the present invention may also be in the form of lacquer, gel, foam, salve, ointment, lotion, solution, or other form suitable for topical delivery, as are known in the art.

The composition of the present invention may also be used in combination with any of a bandage, a patch, a wrap, a dressing, or a similar containment, or delivery, platform that is known in the art.

Although the topically applied composition of the present invention is a novel alternative to an orally administered therapy, it may be used in combination with an orally administered therapy as a co-therapy, without departing from the spirit and scope of the invention.

Although the topically applied composition of the present invention is a novel alternative to a transcutaneous injection therapy, it may be used in combination with a transcutaneous injection therapy as a co-therapy, without departing from the spirit and scope of the invention.

### EXAMPLES

The Examples illustrate preferred embodiments of the invention and are not to be regarded as limiting.

### Anti-Inflammatory Example

The Example in Table 1 below specifically utilizes the anti-inflammatory agent ketoprofen, and the formulation is intended to be used for treating damaged soft tissue, such as is caused by sprains, strains, bruises, etc. The invention matrix is designed for maximum throughput (flux) through the skin so as to reach underlying tissue and muscle, thereby treating the injury with the anti-inflammatory agent. All percentages are by weight.

TABLE 1

| Specific Composition | Amount (%) | General Composition |
|---|---|---|
| Ketoprofen Powder USP | 9.8 | Drug, i.e. anti-inflammatory |
| | | 2% to 20% |
| Cyclopentadecanolide | 11.5 | Proprietary enhancer; |
| (CPE-215 ®) | | 0.5 to 25% |
| Lecithin Soya Granular NF | 9.8 | Gel Forming Agent |
| | | 1% to 25% |
| Reagent Grade Water | 47 | Solvent (as needed up to 80%) |
| Alcohol, 190 proof | Qs | Solvent alcohol (isopropanol |
| | | could be used) |
| Pluronic F127 NF | 11.8 | Surfactant and gel forming |
| | | agent (1% to 20%) |
| Potassium Sorbate NF | 0.001 | Preservative |
| Sorbic Acid NF | 0.0005 | Preservative |

### Anti-Fungal Examples

The Examples in Table 2 below specifically utilize the anti-fungal agents terbinafine and clotrimazole, and the formulations are intended to be used for treating onychomycosis. The invention matrix is designed for maximum throughput (flux) through the nail. All percentages are by weight.

TABLE 2

| | Concentration | Concentration | Formula- | Formulation |
|---|---|---|---|---|
| Drug | of Active | of CPE-215 | tion Type | Number |
| Terbinafine | 1% | 8% | Cream | I |
| Clotrimazole | 1% | 8% | Lacquer | II |
| Terbinafine | 3% | 8% | Lacquer | III |
| Clotrimazole | 4% | 8% | Lacquer | IV |
| Terbinafine | 1% | 8% | Lacquer | V |
| Terbinafine | 5% | 8% | Lacquer | VI |
| Terbinafine | 3% | 8% | Cream | VII |
| Clotrimazole | 1% | 8% | Cream | VIII |

### Terbinafine Cream Preparation:

### Three stock solutions and cottonseed oil are used for making the Terbinafine Cream.

### Ratio of Stock Solutions and Cottonseed Oil:

| | |
|---|---|
| CPE-215/Lecithin Stock | 18.1% |
| Lutrol F127 Stock | 62.1% |
| Drug Stock | 15.8% |
| Cottonseed Oil | 4.0% |
| | 100% |

### Preparation of Stock Solutions:

### CPE-215/Lecithin Stock

| Reagents | w/w % |
|---|---|
| CPE-215 (lig.) | 44.2 |
| Lecithin Granular NF (Soy) (Spectrum | 55.3 |
| L1083) | |
| Sorbic Acid NF(solid) (Spectrum SO215) | 0.24 |
| Lavender Oil (lig.) | 0.28 |
| Total | 100 |

1) Warm CPE-215 to 40 C. to melt it.
2) Weigh into a vessel the CPE-215. Vessel can be HDPE.
3) Weigh Sorbic Acid into the vessel.
4) Weigh Lavender Oil into the vessel.
5) Place vessel into water bath and raise temperature to 75–80° C.
6) Weigh into a weight boat the Lecithin.
7) Mix the CPE-215 with a Cowles blade propeller at high speed.
8) Add the lecithin to the vessel slowly.
9) Continue mixing and maintain temperature at 75–80 C. until homogenous (no lumps).
Note:
A 500 gram batch took 1.5 hours to complete with this method. At the end the material can be transferred from HDPE to glass for storage.

### Lutrol F127 Stock

| Reagent | w/w % |
|---|---|
| Lutrol F127 NF (BASF) | 18.1 |
| Potassium Sorbate NF (Spectrum PO300) | 0.2 |
| Sterile Water | 81.7 |
| Total | 100 |

1) Cool water bath to approximately 5° C.
2) Cool sterile water to 5° C.
3) Weigh Potassium Sorbate and add to vessel.
4) Weigh Lutrol F127 into a weigh boat.
5) Weigh and add the sterile water to vessel. Return vessel to water bath and maintain 5° C. water bath temperature and vessel content temperature.
6) Mix contents of vessel with Cowles blade propeller at high speed.
7) At a moderate speed add the weighed Lutrol F127 into the vessel.
8) Lower the speed of the mixer to 60% and maintain temperature at 5° C.
9) Mix for 40 minutes.
10) Remove from mixer and seal.
11) Let stand in refrigerator or water bath at 5° C. for 1 hour and let bubbles rise to the top and dissipate. Check for undissolved Lutrol F127.

### Drug Stock

| Reagent | w/w % |
|---|---|
| Terbinafine Base (Spectrum YY1084) | 19 |
| Ethanol 200 Proof USP (Pharmco) | 81 |
| Total | 100 |

1) Weigh Terbinafine Base and add to vessel.
2) Weigh and add Ethanol 200 Proof to vessel.
3) Mix until Terbinafine Base is completely dissolved.

### Terbinafine Cream Final Preparation:

| Reagent/Stock | w/w % |
|---|---|
| Lutrol F127 Stock | 62.1 |
| Drug Stock | 15.8 |
| Cottonseed Oil NF (Spectrum CO145) | 4.0 |
| CPE-215/Lecithin Stock | 18.1 |
| Total | 100 |

1) Heat CPE-215/Lecithin Stock to 50–55° C. It will turn from a solid to a liquid.
2) Cool Lutrol F127 Stock to 10–15° C.
3) Weigh and add Cottonseed Oil to the CPE/Lecithin Stock and mix with stir paddle. Use more of the CPE-215/Lecithin Stock for this step as it will stick to the container.
4) In a wide mouth vessel, add the Drug Stock.
5) Mix with a paddle blade propeller. Propeller must be able to move thick material.
6) Weigh and add to the vessel the Lutrol F127 Stock. Terbinafine Base will crystallize out.
7) Weigh and add to the vessel the CPE-215/Lecithin Stock.
8) Mix thoroughly until creamy. Material will thicken as temperature drops.
9) Continue mixing until homogenous.
10) Seal container or place into lacquer lined tubes.
Note:
Next day the cream rings or vibrates when tapped.

### Clotrimazole Lacquer Preparation:

| Reagents: | w/w % |
|---|---|
| A) Clotrimazole USP | 1 |
| B) CPE-215 | 8 |
| C) Propylene Glycol | 5 |
| D) Ethanol 190 Proof | 82 |
| E) Eudragit RL PO | 4 |
| | 100 |

Procedure:
1) Warm CPE-215 to 40° C. to melt it.
2) Weigh Clotrimazole and place in a vessel.
3) Weigh and add Propylene Glycol to the vessel.
4) Weigh and add CPE-215 to the vessel.
5) Weigh and add Ethanol 190 Proof to the vessel.
6) Mix until drug and CPE-215 is dissolved.
7) Weigh and add Eudragit RL PO to the vessel while mixing. Eudragit may clump up but will dissolve completely.
Note:
CPE-215 is Cyclopentadecanolide or Pentadecalactone

### DESCRIPTION OF THE DRAWINGS

FIG. 1 depicts the results of 10 μl applications of each of the anti-fungal formulations in Table 2 against the test organism T. rubrum through 5 μm thickness of human nail. The Application rate is 10 mg/cm². The hatched area indicates the maximum length of zone of inhibition has been reached (e.g. total kill in the test system and no measurable zone). Variations between the zones of inhibition are only indicative of variations in TurChub® cell lengths.

FIG. 2 depicts the results of 2 μl applications of four anti-fungal formulations (III, V, VI, and VII) in Table 2 against the test organism T rubrum through 5 μm thickness of human nail. The Application rate is 2 mg/cm². In the 10 μapplications depicted in FIG. 1 these four anti-fungal formulations (III, V, VI, and VII) had reached or were close to the maximum length of zone of inhibition. The hatched area in FIG. 2 indicates the maximum length of zone of inhibition has been reached (e.g. total kill in the test system and no measurable zone).

### DISCUSSION OF THE 10 μl APPLICATION DATA

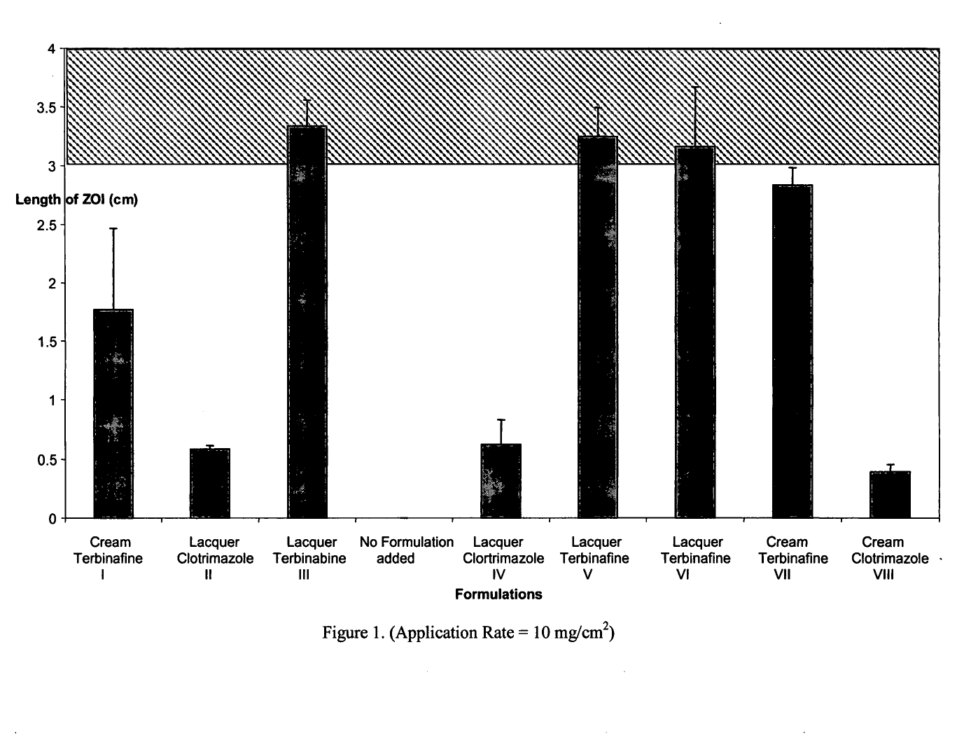

All results presented as average length of zone of inhibition (cm) where n=5± standard deviation against the test organism T.rubrum through 5 μm thickness human nail. The hatched area in FIG. 1 indicates the maximum length of zone of inhibition has been reached (e.g. total kill in the test system and no measurable zone).

Statistical analysis of results: One-way ANOVA - 10 μl application

| Source | DF | SS | MS | F | P |
|---|---|---|---|---|---|
| C1 | 8 | 67.265 | 8.408 | 75.92 | 0.000 |
| Error | 33 | 3.655 | 0.111 | | |
| Total | 41 | 70.920 | | | |

S = 0.3328
R-Sq = 94.85%
R-Sq(adj) = 93.60%

| | | | | Individual 95% CIs For Mean Based on |
|---|---|---|---|---|
| | | | | Pooled StDev |
| Formulation | N | Mean | StDev | ----+---------+---------+---------+----- |
| I | 5 | 1.7700 | 0.6967 | (--*-) |
| VII | 5 | 2.8360 | 0.1477 | (--*-) |
| VIII | 5 | 0.3960 | 0.0611 | (-*--) |
| II | 5 | 0.5860 | 0.0297 | (--*-) |
| V | 5 | 3.2520 | 0.2414 | (-*--) |
| III | 5 | 3.3400 | 0.2162 | (--*-) |
| IV | 5 | 0.6220 | 0.2075 | (-*--) |
| VI | 5 | 3.1620 | 0.5037 | (-*--) |
| No Formulation | 2 | 0.0000 | 0.0000 | (---*---) |
| | | | | ----+---------+---------+---------+----- |
| | | | | 0.0 1.2 2.4 3.6 |

Pooled StDev = 0.3328

Ranking with 10 μl application (according to ANOVA statistical analysis):
1) III≧V≧VI≧VII
2) I
3) VI≧II≧VIII

The zones of inhibition which are in the hatched area, have reached the maximum length of zone of inhibition and variations between the zones of inhibition are only indicative of variations in TurChub® cell lengths. The four formulations that had reached or were close to the maximum length of zone of inhibition were repeated using only a 2 μl application of the formulation to allow comparison of these formulations.

### DISCUSSION OF THE 2 μl APPLICATION DATA

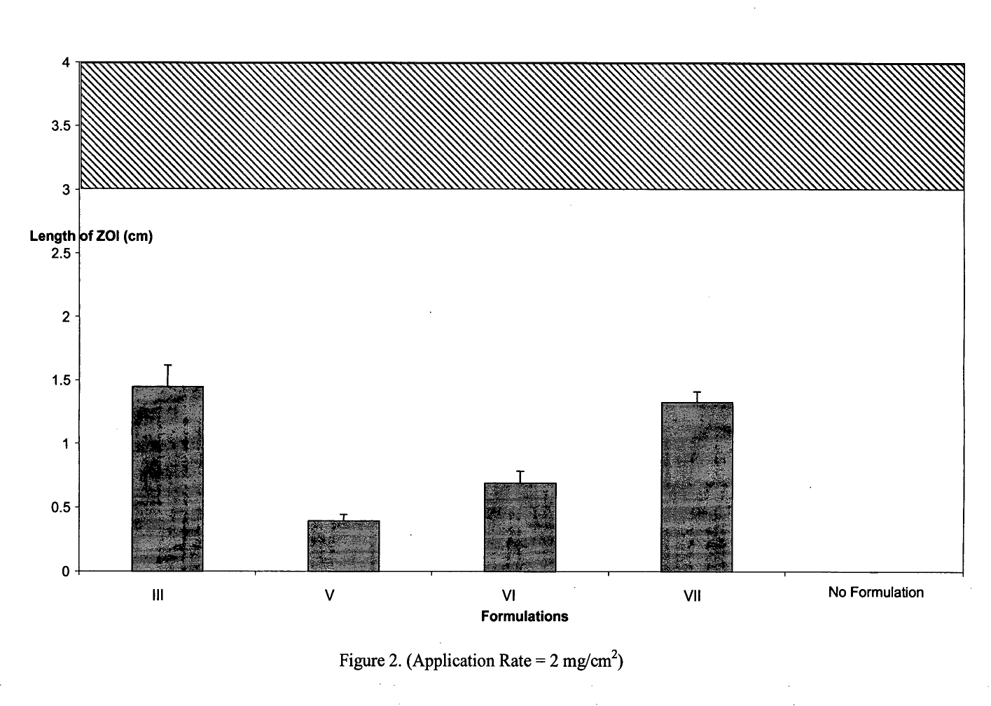

All results presented as average length of zone of inhibition (cm) where n=5± standard deviation against the test organism T.rubrum through 5 μm thickness human nail. The hatched area in FIG. 2 indicates the maximum length of zone of inhibition that could be reached, the zones of inhibition are only indicative of variations in TurChub® cell lengths.

Statistical analysis of results: One-way ANOVA - 2 μl application

| Source | DF | SS | MS | F | P |
|---|---|---|---|---|---|
| C1 | 4 | 5.5312 | 1.3828 | 124.93 | 0.000 |
| Error | 17 | 0.1882 | 0.0111 | | |
| Total | 21 | 5.7193 | | | |

S = 0.1052
R-Sq = 96.71%
R-Sq(adj) = 95.94%

| | | | | Individual 95% CIs For Mean Based on |
|---|---|---|---|---|
| | | | | Pooled StDev |
| Formulation | N | Mean | StDev | ---+---------+---------+---------+------ |
| VII | 5 | 1.3260 | 0.0847 | (-*-) |
| V | 5 | 0.3920 | 0.0512 | (-*-) |
| III | 5 | 1.4480 | 0.1684 | (-*-) |
| VI | 5 | 0.6880 | 0.0942 | (-*-) |
| No Formulation | 2 | 0.0000 | 0.0000 | (--*--) |
| | | | | ---+---------+---------+---------+------ |
| | | | | 0.00 0.50 1.00 1.50 |

Pooled StDev = 0.1052

Ranking with 2 μl application (according to ANOVA statistical analysis):
1) III≧VII
2) VI
3) V

According to the statistical analysis, formulations III and VII showed the highest efficacy against T. rubrum through 5 μm thickness human nail.

We claim:

1. A topically applied pharmaceutical composition for enhancing any of the transdermal, subdermal, transmembrane, musculoskeletal, transungual, transonychial, or peronychial deliveries of a therapeutically effective amount of an active ingredient to a patient, said composition comprising:
(a) an active ingredient,
(b) an organogel, and
(c) a non-irritating permeation enhancer used as a structural substituent of the organogel.

2. The topically applied pharmaceutical composition of claim 1, wherein said active ingredient is an antifungal agent.

3. The topically applied pharmaceutical composition of claim 2, wherein said antifungal agent is suitable for treating onychomycosis.

4. The topically applied pharmaceutical composition of claim 3, wherein said antifungal agent is selected from the group consisting of itraconazole, clotrimazole, variconazole, ciclopirox, and terbinafine.

5. The topically applied pharmaceutical composition of claim 4, wherein said antifungal agent is terbinafine

6. The topically applied pharmaceutical composition of claim 1, wherein said active ingredient is an anti-inflammatory agent.

7. The topically applied pharmaceutical composition of claim 6, wherein said anti-inflammatory agent is an NSAID.

8. The topically applied pharmaceutical composition of claim 7, wherein said NSAID is ketoprofen.

9. The topically applied pharmaceutical composition of claim 7, wherein said NSAID is a cyclooxygenase Type 2 inhibitor.

10. The topically applied pharmaceutical composition of claim 1, wherein said non-irritating permeation enhancer is a Hsieh-type enhancer.

11. The topically applied pharmaceutical composition of claim 10, wherein said Hsieh-type enhancer is cyclopentadecalactone or cylcohexadecanone.

12. The topically applied pharmaceutical composition of claim 10, wherein said Hsieh-type enhancer is cyclopentadecanolide.

13. The pharmaceutical composition of claim 1, wherein said non-irritating permeation enhancer is a straight chain or branched compound of the saturated or unsaturated aliphatic type.

14. The pharmaceutical composition of claim 13, wherein said straight chain or branched compound of the saturated or unsaturated aliphatic type is selected from the group consisting of: myristyl myristate, octyl palmitate, and ethyl oleate.

15. The pharmaceutical composition of claim 14, wherein said straight chain or branched compound of the saturated or unsaturated aliphatic type is selected from the group consisting of: myristyl myristate, octyl palmitate, and ethyl oleate used in combination with the Hsieh type enhancer of claim 9.

16. The pharmaceutical composition of claim 14, wherein said straight chain or branched compound of the saturated or unsaturated aliphatic type is myristyl myristate: myristyl myristate or octyl palmitate used in combination with cyclopentadecalactone.

17. A topically applied pharmaceutical composition for treating a patient for onychomycosis, said composition comprising:
(a) an active ingredient,
(b) an organogel, and
(c) a non-irritating permeation enhancer.

18. The topically applied pharmaceutical composition of claim 17, wherein said active ingredient is terbinafine.

19. A method of treating a patient with an active ingredient, said method comprising topically applying the pharmaceutical composition of claim 1.

20. A method of treating a patient with an active ingredient, said method comprising topically applying the pharmaceutical composition of claim 2.

21. A method of treating a patient with an active ingredient, said method comprising topically applying the pharmaceutical composition of claim 3.

22. A method of treating a patient with an active ingredient, said method comprising topically applying the pharmaceutical composition of claim 4.

23. A method of treating a patient with an active ingredient, said method comprising topically applying the pharmaceutical composition of claim 5.

24. A method of treating a patient with an active ingredient, said method comprising topically applying the pharmaceutical composition of claim 6.

25. A method of treating a patient with an active ingredient, said method comprising topically applying the pharmaceutical composition of claim 7.

26. A method of treating a patient with an active ingredient, said method comprising topically applying the pharmaceutical composition of claim 8.

27. A method of treating a patient with an active ingredient, said method comprising topically applying the pharmaceutical composition of claim 9.

28. A method of treating a patient with an active ingredient, said method comprising topically applying the pharmaceutical composition of claim 10.

29. A method of treating a patient with an active ingredient, said method comprising topically applying the pharmaceutical composition of claim 11.

30. A method of treating a patient with an active ingredient, said method comprising topically applying the pharmaceutical composition of claim 12.

31. A method of treating a patient with an active ingredient, said method comprising topically applying the pharmaceutical composition of claim 13.

32. A method of treating a patient with an active ingredient, said method comprising topically applying the pharmaceutical composition of claim 14.

33. A method of treating a patient with an active ingredient, said method comprising topically applying the pharmaceutical composition of claim 15.

34. A method of treating a patient with an active ingredient, said method comprising topically applying the pharmaceutical composition of claim 16.

35. A method of treating a patient with an active ingredient, said method comprising topically applying the pharmaceutical composition of claim 17.

36. A method of treating a patient with an active ingredient, said method comprising topically applying the pharmaceutical composition of claim 18.

37. A topically applied pharmaceutical composition for enhancing any of the transdermal, subdermal, transmembrane, musculoskeletal, transungual, transonychial, or peronychial deliveries of a therapeutically effective amount of an active ingredient to a patient, said composition comprising:
(a) an active ingredient selected from the group consisting of acyclovir, famciclovir, penciclovir, and valacyclovir,
(b) an organogel, and
(c) a non-irritating permeation enhancer used as a structural substituent of the organogel.

38. A method of treating a patient with an active ingredient selected from the group consisting of acyclovir, famciclovir, penciclovir, and valacyclovir, said method comprising topically applying the pharmaceutical composition of claim 37.
